(19) United States
(12) Patent Application Publication
(10) Pub. No.: US 2022/0254500 A1
(43) Pub. Date: Aug. 11, 2022

(54) SYSTEMS AND METHODS FOR DETECTION AND GRADING OF DIABETIC RETINOPATHY

(71) Applicant: University of Louisville Research Foundation, Inc., Louisville, KY (US)

(72) Inventors: AYMAN S. EL-BAZ, Louisville, KY (US); HARPAL SANDHU, Louisville, KY (US); ROBERT S. KEYNTON, Goshen, KY (US)

(21) Appl. No.: 17/639,825

(22) PCT Filed: Sep. 4, 2020
(86) PCT No.: PCT/US20/49502

§ 371 Date: Mar. 2, 2022

Related U.S. Application Data
(60) Provisional application No. 62/897,048, filed on Sep. 6, 2019.

(51) Int. Cl. G16H 50/30; G16H 30/40; G16H 10/60; A61B 5/00; A61B 5/1455; G06T 7/00

(52) U.S. Cl. CPC G16H 50/30; G16H 30/40; G16H 10/60; G06T 2207/30041; A61B 5/1455; G06T 7/0012; G06T 2207/10101; A61B 5/4842

(57) ABSTRACT

Computer-implemented systems and methods for automated diagnosis of diabetic retinopathy apply machine learning techniques to clinical and demographic data combined with optical coherence tomography and optical coherence tomography angiography image data to diagnose and grade diabetic retinopathy.

This application claims the benefit of U.S. provisional patent application Ser. No. 62/897,048 filed 6 Sep. 2019 for SYSTEMS AND METHODS FOR DETECTION AND GRADING OF DIABETIC RETINOPATHY, incorporated herein by reference.

### FIELD OF THE INVENTION

Computer-implemented systems and methods for automated diagnosis of diabetic retinopathy apply machine learning techniques to clinical and demographic data combined with optical coherence tomography and optical coherence tomography angiography image data to diagnose and grade diabetic retinopathy.

### BACKGROUND OF THE INVENTION

Diabetic retinopathy (DR) is a complication of diabetes mellitus (DM), which can lead to blindness. DR is considered one of the major causes of blindness worldwide. DR progresses from mild nonproliferative DR (NPDR) to moderate NPDR to severe NPDR to proliferative DR (PDR). 40% of patients with DR have some degree of diabetic macular ischemia (DMI). DMI is characterized by foveal avascular zone (FAZ) enlargement and the existence of a parafoveal area of capillary dropout. The progression of DMI has been linked to visual acuity, which is essential in DR recognition. DR is recognized by microaneurysms, capillary drop-out, and ischemia. DR may give rise to some complexities like DMI and diabetic macular edema (DME). The capillary dropout reduces the nutrition of the tissues in the retina, causing a rise in the vascular endothelial growth factor, which causes vascular permeability and angiogenic responses. In summary, changes, like vessel dilation and tortuosity, microaneurysms, capillary dropout, and FAZ enlargement, begin to appear as DR is developing.

Ophthalmologists can avoid this vision loss by detecting DR in its early stages. There is a need for imaging modalities that can show the changes that occur in the retinal blood vasculature and layers. Fluorescein angiography (FA) is the standard imaging modality used for the ocular vasculature and for the diagnosis of macular perfusion. FA involves the injection of dye followed by a serial of fundus imaging. FA is invasive, costly, time-consuming, cannot be used frequently, and has many undesirable side effects. Some of the less serious side effects of FA include nausea, vomiting, yellow pigmentation of the skin, and discolored urine. More severe effects include anaphylactoid reactions ranging from skin rash and itching to severe anaphylactic shock, which provides a small risk of severe bronchospasm and death. A serious limitation of FA technique is the leakage of dye from the blood vessels.

Optical coherence tomography (OCT) is an emerging imaging technique in diagnosing eye diseases, which has been comprehensively utilized for inspecting the anterior segment of the human's eye, including diagnosis of corneal disorders. Many studies have used OCT images in classifying and detecting DR. The only objective data that OCT currently provides are crude measurements of thickness like central macular volume (CMV) and central macular thickness (CMT), which are values determined by OCT that do not correlate well with visual acuity or with leakage observed by FA. It does not provide any information about the retinal vasculature network.

Optical coherence tomography angiography (OCTA) is a noninvasive imaging modality, which produces retinal vasculature network images. It compares the decorrelation signal between multiple consecutive optical coherence tomography (OCT) B-scans captured at the same cross-section. OCTA provides the ophthalmologist with detailed images of the retinal vasculature in deep, superficial, and capillary plexuses. OCTA provides a way to observe the ischemic changes that impact different plexuses of the retina. For example, superficial retinal plexus (SRP) can be affected by cotton wool spots, whereas paracentral acute middle maculopathy affects deep retinal plexus (DRP). OCTA can provide detailed perfusion information and anatomic details that assist in the prediction of different ophthalmic diseases. For example, Tarassoly et al. experimented to see the capability of OCTA in pointing out the abnormalities in DR patient's images and compared it with FA. Ishibazawa et al. evaluated how OCTA images can capture the features of DR to detect microaneurysms, neovascularization, and retinal nonperfused areas in DR patients. Bhanushali et al. used OCTA images to extract features that can differentiate between DR grades. They noticed that DR patients have larger FAZ area and lower vessel density than normal cases. Mild and moderate NPDR have lower spacing between large vessels than PDR and severe NPDR.

The limitations of current work of DR can be summarized into the following points. First, most of the current work has focused on the detection of lesions in DR patients. Few of these studies have gone further and used non-clinical features to detect DR. Second, the majority of the current work was interested only in studying layers of the retina using OCT images regardless of the changes that occur in the blood vessels, demographic data, or clinical data. Third, the subjective interpretation by a retina specialist is a significant limitation of OCT and OCTA technologies that limit access and delay DR diagnosis and treatment. Finally, no know system integrates OCT and OCTA features with demographic and clinical data. This kind of integration between these various features can help to provide a comprehensive CAD system that has the ability to provide a precise diagnosis for DR.

### SUMMARY

To address these limitations, the inventors present an objective computer-aided diagnostic (CAD) system that integrates OCT and OCTA data imaging with patients' clinical data and demographic data using machine learning techniques to detect and grade early stages of DR. First, a plurality of retinal layers are extracted from the OCT scan. Next, the retinal vasculature network is extracted from two different OCTA plexuses, which are SRP and DPR. Then, significant retinal features are extracted, which reflect the changes in the retinal blood vessels and retinal layers due to DR progress. Extracted features from OCT include thickness, curvature, and reflectivity of each retinal layer. Extracted features from OCTA include the retinal vasculature network for determination of bifurcation and crossover points, vascular density, vessel caliber, and the area of the foveal avascular zone (FAZ). Classification uses a two-stage, cascaded random forest (RF) based approached. First, the classifier differentiates normal from DR subjects. Second, the classifier differentiates between grades of DR. In the experimental results, the system achieved an average ACC of 97%, which outperforms other state-of-the-art techniques.

While many studies have applied machine learning, including deep learning, to fundus photographs to diagnose DR, OCT and OCTA have rarely been focused on, and never in combination with demographic data and clinical markers as in the disclosed invention. Multiple groups have applied machine learning to OCT to identify macular edema of various etiologies. Automated systems for diagnosis of exudative age-related macular degeneration (AMD) and geographic atrophy on OCT have also shown good results. One deep learning system trained on over 14,000 OCT images in the United Kingdom can provide probabilities of ten different common OCT diagnoses and produced a correct referral recommendation, the primary outcome, 95% of the time. However, it also simply identified macular edema rather than diagnosing a particular etiology of edema, and DR itself was not one of the ten output diagnoses.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 is a flowchart illustrating a computer-implement method for diagnosing and grading DR.

FIG. 2 is a graph depicting the different LCDG models for the retinal layers from OCT scan wherein the X-axis is the intensity values and the y-axis is the probability density.

FIG. 3 depicts a probabilistic color map of the retinal layers, different colors representing different retinal layers.

FIG. 4 depicts the thickness of retinal layers for normal (left), mild NPDR (center), and moderate NPDR (right).

FIG. 5 depicts the retinal layer reflectivity for normal (left), mild NPDR (center), and moderate NPDR (right).

FIG. 6 depicts the retinal curvature for normal (left), mild NPDR (center), and moderate NPDR (right).

FIG. 7A depicts an OCTA image, a segmented OCTA image, and a CDF graph of blood vessel density for a normal retina.

FIG. 7B depicts an OCTA image, a segmented OCTA image, and a CDF graph of blood vessel density for a mild NPDR retina.

FIG. 7C depicts an OCTA image, a segmented OCTA image, and a CDF graph of blood vessel density for a mild NPDR retina.

FIG. 8 depicts blood vessel caliber for normal, mild NPDR, and moderate NPDR.

FIG. 9 depicts FAZ distance maps for normal, mild NPDR, and moderate NPDR.

FIG. 10 depicts bifurcation and crossover points for normal, mild NPDR, and moderate NPDR.

FIG. 11 is a graph displaying the accuracy of various feature combinations using various classifiers. For each feature, the five columns, left to right, indicate RF, SVM Linear, SVM Cubic, KNN, and CT classifiers.

FIG. 12 is a graph displaying the Dice Similarity Coefficient (DSC) of various feature combinations using various classifiers. For each feature, the five columns, left to right, indicate RF, SVM Linear, SVM Cubic, KNN, and CT classifiers.

FIG. 13 is a graph displaying the ROC curve for RF classifier in the detection stage.

FIG. 14 is a graph displaying the ROC curve for RF classifier in the grading stage.

### DETAILED DESCRIPTION OF THE PREFERRED EMBODIMENTS

For the purposes of promoting an understanding of the principles of the invention, reference will now be made to selected embodiments illustrated in the drawings and specific language will be used to describe the same. It will nevertheless be understood that no limitation of the scope of the invention is thereby intended; any alterations and further modifications of the described or illustrated embodiments, and any further applications of the principles of the invention as illustrated herein are contemplated as would normally occur to one skilled in the art to which the invention relates. At least one embodiment of the invention is shown in great detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to “advantages” provided by some embodiments of the present invention, other embodiments may not include those same advantages, or may include different advantages. Any advantages described herein are not to be construed as limiting to any of the claims.

Specific quantities (spatial dimensions, dimensionless parameters, etc.) may be used explicitly or implicitly herein, such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated. Unless stated otherwise, explicit approximate quantities (e.g., about 1; approximately 20) refer to a range of ±5% of the recited quantities (e.g., “about 1” refers to 0.95 to 1.05; “approximately 20” refers to the range of 19 to 21). The terms “extract” and “segment” are used interchangeably herein (e.g., extracting the blood vasculature network and segmenting the blood vasculature network refer to the same process).

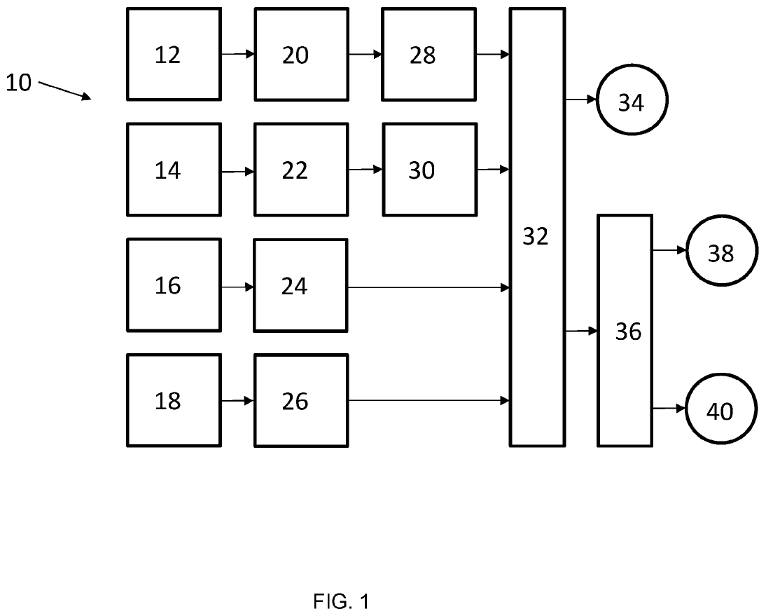

Disclosed herein is a novel comprehensive system and computer-aided method for early detection of DR as well as the detection of different DR grades. The proposed system is based on the analysis of OCT and OCTA scans, along with the patient's clinical and demographic data, using machine learning techniques to objectively classify a subject retina. Referring to FIG. 1, the system 10 includes 12—receiving OCT image data from one or more OCT scans of a subject retina of an individual, 14—receiving OCTA image data from one or more OCTA scans of the subject retina of the individual, 16—receiving demographic data of the individual, and 18—receiving clinical marker data of the individual, 20—preprocessing the OCT image data to enhance image contrast and remove noise, and segmenting the retinal layers from the OCT image data, 22—preprocessing the OCTA image data to enhance image contrast and remove noise, and segmenting the blood vascular network from two different capillary plexuses, namely, the superficial vascular plexus (SVP) and deep vascular plexus (DVP), 24—preprocessing the demographic data to normalize values and impute missing values, 26—preprocessing the clinical data to normalize values and impute missing values, 28—extracting features from the segmented OCT image data including, in some embodiments, extracting from each retinal layer the retinal layer curvature, reflectivity, and thickness, 30—extracting features from the segmented OCTA image data including, in some embodiments, extracting the blood vessels caliber, FAZ, bifurcation and crossover points, and the blood vessels density, 32—differentiation of DR from normal cases (34) using machine learning techniques, in some embodiments, using a random forest (RF) approach, and 36—distinguishing mild NPDR (38) and moderate NPDR 40) using the machine learning techniques. The final stages of DR diagnosis and grading 32, 36, can be considered a two-stage RF classification, wherein the first stage is responsible for the detection of DR and differentiating it from normal cases and the second stage is implemented to distinguish mild from moderate NPDR. While OCT image data, OCTA image data, demographic data, and clinical marker data may be received or otherwise obtained using techniques generally known in the art, each of the other steps is described in further detail.

Retina Layer Segmentation

The disclosed retinal layer segmentation approach is used to detect twelve layers from OCT scans. The segmentation approach utilized a comprehensive model that integrates spatial, shape, and appearance information. An input 2-dimensional (2D) OCT image, with integer intensity gray values g={g(x): x∈R², g∈|0,255|}, is co-registered to an atlas (training database), and its map L, which is a group of region labels, as explained with a joint probability model:

P(g;L)=P(g|L)P(L) (1)

The model integrates a conditional probability distribution P(g|L) of the images (g) by providing the map (L) and an unconditional distribution of maps P(L)=Pₛₚ(L)P_{V}(L). Pₛₚ(L) describes a weighted shape prior, whereas P_{V}(L) denotes probability density function of Gibbs distribution with potentials (V), which presents a Markov-Gibbs random field (MGRF) probability model. The layer segmentation approach is generated as a joint probability of the following models.

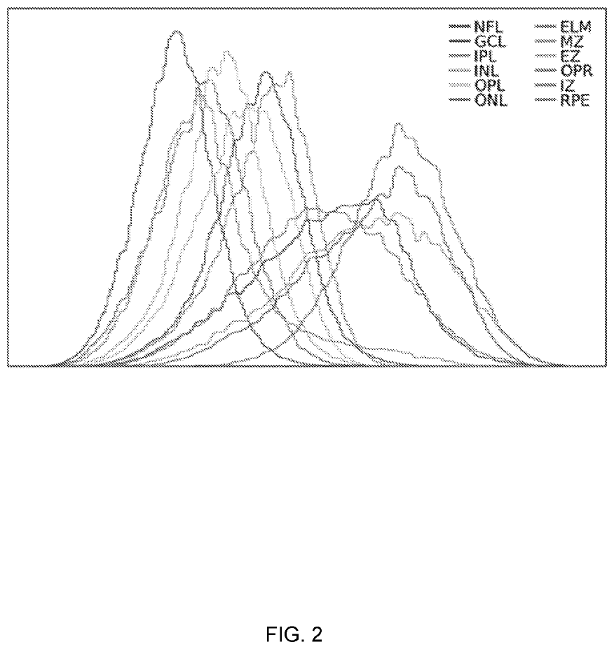

1st-Order Appearance Model P(g|L): The brightness of distinct labels in the image is represented using the first-order visual model by distributing the pixel reflectivities into separate components (FIG. 2). These components are combined with the dominant modes of the mixture. This operation is done utilizing a linear combination of discrete Gaussian (LCDG) approach with positive and negative Gaussian components. LCDG can be considered as a modified version of the common Expectation-Maximization (EM) approach. For complete explanation and details of the LCDG and the revised EM algorithm, see El-Baz, A. & Gimelfarb, G. Em based approximation of empirical distributions with linear combinations of discrete gaussians. In 2007 IEEE International Conference on Image Processing, vol. 4, 373-376, DOI: 10.1109/ICIP.2007.4380032 (2007), incorporated herein by referenced.

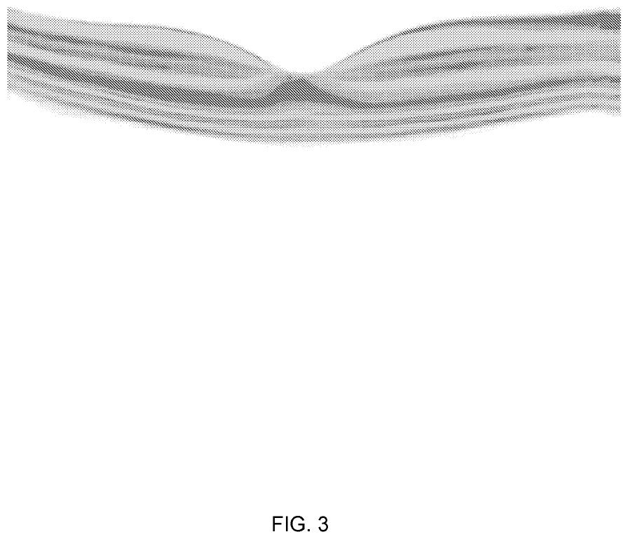

Adaptive Shape Model Pₛₚ(m): In this model, a set of OCT images is used to acquire the biological changes of the DR retina as compared to a normal retina. Using one optimal (i.e., high quality, not blurred or twisted) scan as a reference, the remaining scans were co-aligned using a thin plate spline. This model was also presented to its respective manual segmentation (ground truth (GT)). Consequently, it was standardized by averaging a probabilistic shape prior (atlas) of the healthy retinal layers (FIG. 3).

$${P}_{sp} ( L ) = {\prod }_{y ∈ {R}^{2}} {p}_{sp : y} ( l ) \qquad ( 2 )$$

where Pₛₚ(L) defines the weighted shape prior, p_{sp:y}(l) is the pixel-wise probability for label l, and y is the image pixel.

2nd-Order Spatial Model P_{V}(m): The MGRF Potts model, which takes into consideration spatial information, was merged with the appearance and shape information. To identify such MGRF model, the closest 8-pixels were used as a neighborhood ns system and analytical bi-valued Gibbs potentials as:

$$p ( L ) ∝ exp ( {\sum }_{( \sum , y ) ∈ R} {\sum }_{( ξ , ζ ) ∈ {V}_{s}} V ( {l}_{x , y} , {l}_{x + ξ , y + ζ} ) ) \qquad ( 3 )$$

where V is the Gibbs potential values for the current pixel. The process of segmentation of the subject retina in OCT images is explained in detail in Tanboly, A. E. et al. A novel automatic segmentation of healthy and diseased retinal layers from oct scans. In 2016 IEEE International Conference on Image Processing (ICIP), 116-120, DOI: 10.1109/ICIP.2016.7532330 (2016).

Retinal Blood Vessels Segmentation

This stage aims to segment the retinal blood vasculature network from the OCTA scan by using both SRP and DRP. Before segmentation, the OCTA plexuses are preprocessed to enhance homogeneity and reduce noise. First, regional dynamic histogram equalization (RDHE) is applied to OCTA images for uniformly distributing the gray levels in these images. Then, an unsupervised approach, which integrates an adaptive estimation of a gray level threshold with a generalized Gauss-Markov random field (GGMRF) model, is used to improve the OCTA homogeneity.

To segment the retinal vasculature, a joint MGRF model segmentation technique is used which integrates three models. These models are current appearance, prior intensity, and 3D-MGRF spatial models. The current appearance model is calculated to present the current 1st intensity model of the SRP and DRP by using an LCDG. LCDG is implemented to compute the marginal probability distributions for both blood vasculature and background. The prior model is calculated using the gray intensity values from a plurality of OCTA images, which are labeled by three retinal ophthalmologists. A k-nearest neighbor (KNN) technique is then used to estimate the prior probabilities of both blood vessels and background. Finally, the 3D-MGRF spatial model is developed to enhance the results of the segmentation by using a Markov-Gibbs model of region maps. These region maps deemed only pairwise interactions between each region label and its neighbors from 3D OCTA volume that contains both SRP and DRP. A detailed description of the blood vasculature network segmentation technique can be found in Eladawi, N. et al. Early diabetic retinopathy diagnosis based on local retinal blood vessels analysis in optical coherence tomography angiography (octa) images. Med. physics (2018).

Feature Extraction

This stage aims to pull out a set of features from the segmented scans that can be used in the diagnosis stage. Seven features were pulled out from both segmented OCTA and OCT scans. For OCTA, four features were extracted, which are bifurcation and crossover points, distance map of the FAZ, blood vessel density, and blood vessel caliber. For OCT, three features are calculated from the segmented twelve layers of the retina, which are retinal layer thickness, reflectivity, and curvature. In addition to the OCTA and OCT features, seven demographic and clinical biomarkers are preprocessed and normalized to be included in the extracted features. In the next subsections, the extracted features will be presented in more detail.

OCT Feature Extraction

The anatomy of retinal layers is used to detect and measure retinal irregularity. The segmented OCT images can provide various quantitative measures to distinguish retinal morphology. In some embodiments, the features of thickness, reflectivity, and curvature were extracted from OCT scans and computed for each segmented layer.

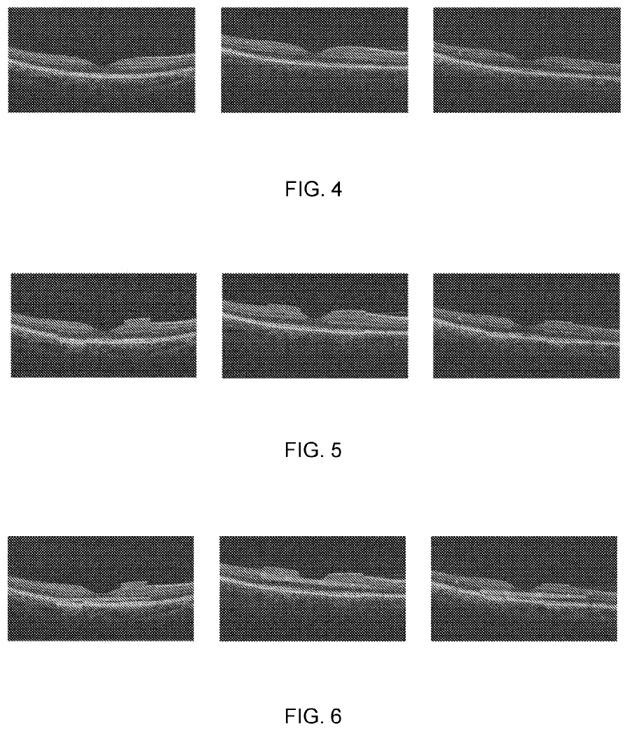

Retinal Thickness: Changes in retinal thickness is indicative of the development of several diseases including retinal vein occlusion (RVO), AMD, and macular edema (ME). The thickness change due to the existence of fluid inside the retina can help in direct clinical decisions regarding medical treatment. In addition, optic disc anatomy and the thickness of retinal nerve fiber layer (RNFL) can track the progression and quantitively measure quantitatively the treatment reaction in glaucoma patients. The thickness of each layer is measured by calculating the shortest Euclidean distances between the upper and lower boundaries of each layer across all points on the boundaries (see FIG. 4, depicting OCT images from normal (left), mild NPDR (middle), and moderate NPDR (right) retinas). The planar Laplace equation: ∇²h=∂²h/∂x²+∂²h/∂y²=0 is solved to match the boundaries points for each segmented layer. h(x;y) is a scalar harmonic function. After solving for h, its gradient vectors induce the streamlines linking the equivalent upper and lower boundaries' points. Finally, the distance between every two equivalent pixels is measured by using Euclidean distance.

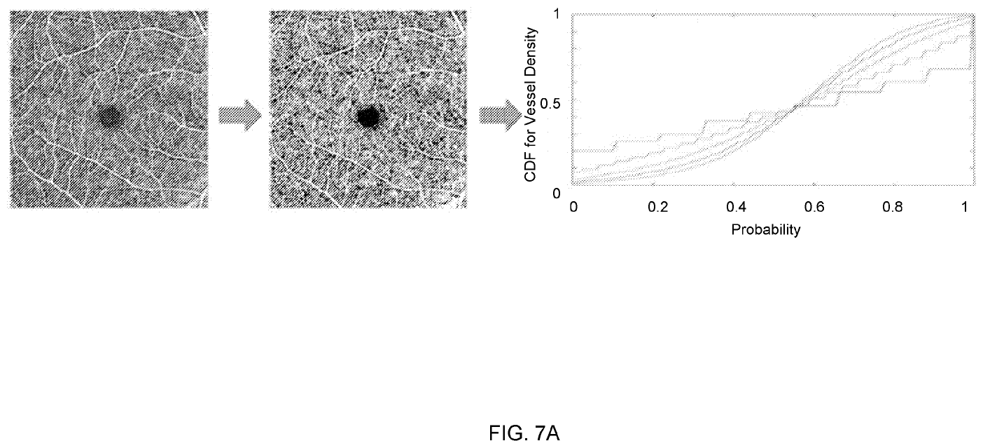

Layer Reflectivity: Retinal layer reflectivity varies significantly by age and between sexes. By incorporating demographic data into the classifier, as described below, layer reflectivity can be normalized against the subject's age and sex, and certain variations from the normalized “norm” indicate DR. The reflectivity (average intensity) in each segmented layer is measured using Huber's M-estimates from two regions per scan, including the thickest portions inside the central foveal region on the temporal and nasal both sides of the fovea (see FIG. 5, depicting OCT images from normal (left), mild NPDR (middle), and moderate NPDR (right) retinas). An advantage of using Huber's M-estimates is its robustness to possible out range values, such as extra bright pixels in the interior segment that belongs to the internal limiting membrane (ILM), rather than the nerve fiber layer (NFL).

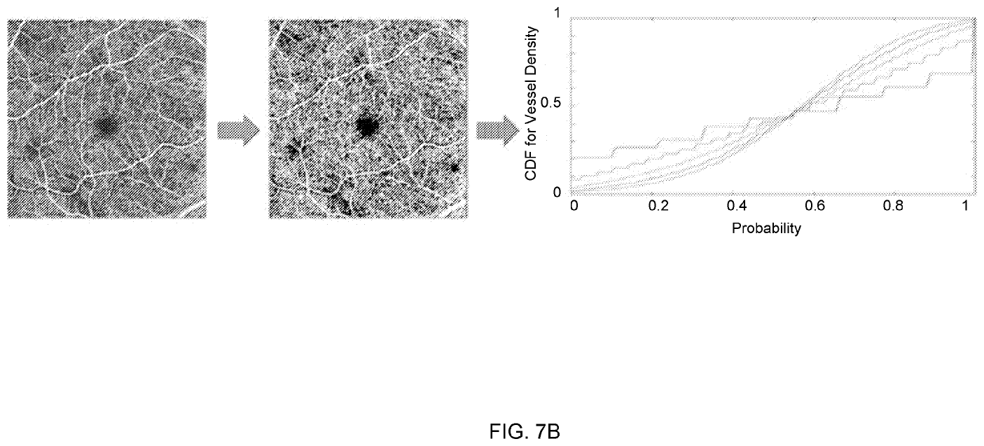

Retinal Layer Curvature: Retinal layer curvature accumulates Menger curvature values measured for each location across the layer after using a locally weighted polynomial of the surface (see FIG. 6, depicting OCT images from normal (left), mild NPDR (middle), and moderate NPDR (right) retinas).

These three extracted features (retinal thickness, layer reflectivity, and retinal layer curvature) are represented as cumulative distribution functions (CDFs) to be fed to the classifier to differentiate between healthy and DR cases. In other embodiments, additional or alternative features may be extracted from the OCT image data.

OCTA Features

Four features were elicited from the segmented OCTA scans to differentiate between normal and DR cases. In some embodiments, these features are bifurcation and crossover points, blood vessel caliber, the distance map of FAZ, and blood vessel density.

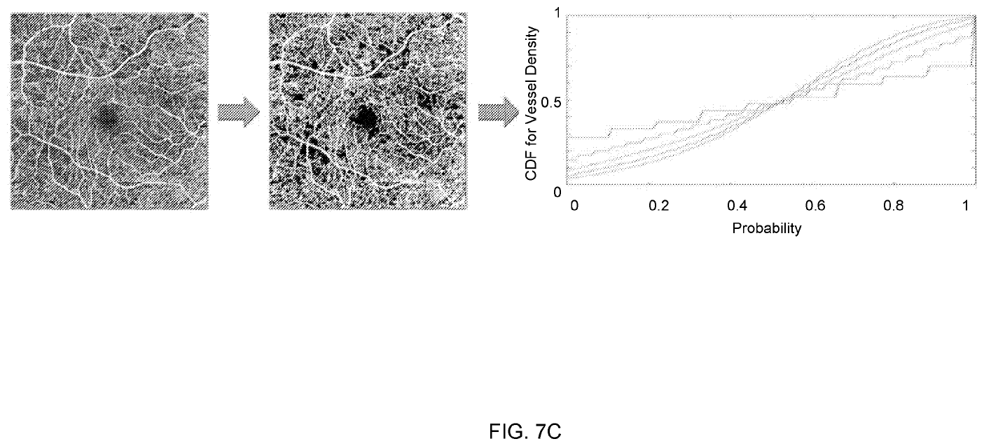

Blood Vessel Density: Blood vessel density in the retina, as captured from segmented OCTA image data, can be used to distinguish between the normal and DR retina. Blood vessel density was extracted from both SRP and DRP using a Parzen window (PW) technique. PW utilizes a given window size to calculate the density (P_{PW}(Bᵣ)) at a specific location r in the segmented image (Bᵣ) depending on the neighbors of the central pixel in this window. Blood vessel density was calculated using various window sizes (3×3, 5×5, 7×7, 9×9, and 11×11) to ensure that the extracted density is not affected by choice of the window size. For each tested window size, a CDF was used to represent these density values as a feature that can be fed to the classifier. In one embodiment, an incremental value of 0.01 was used for the CDFs to be a 100 elements vector. Then, these vectors are fed to the classifier. Referring now to FIG. 7A, the leftmost image depicts an original OCTA image of a normal retina, the central image depicts the segmented OCTA image, and the rightmost graph depicts the resulting CDF, each line representing a different window size. FIGS. 7B and 7B depict similar elements for mild NPDR and moderate NPDR retinas, respectively.

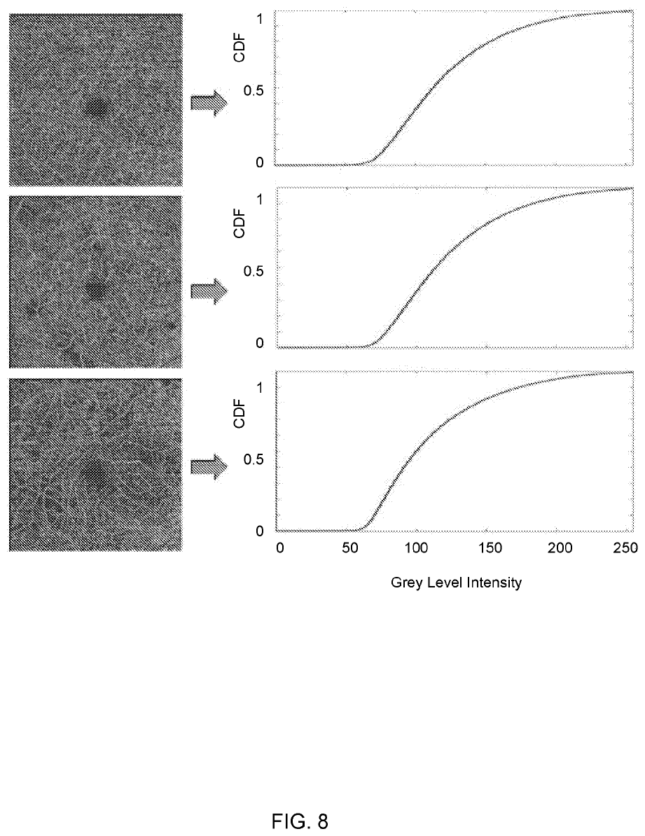

Blood Vessel Caliber: Blood vessel caliber, i.e., diameter, is calculated to differentiate small from large blood vessels using appearance and intensity level. First, the original image was multiplied by the segmented image g for both SRP and DRP. Then, a CDF is created for each gray scale level. These CDFs identify the differences in retinal blood vessel caliber. In some embodiments, an incremental value of 0.02 was used for these CDFs to be represented as vectors of 128 values. FIG. 8 shows blood vessels caliber, as indicated by color, and CDF curves for normal cases (top), mild NPDR cases (middle), and moderate NPDR cases (bottom).

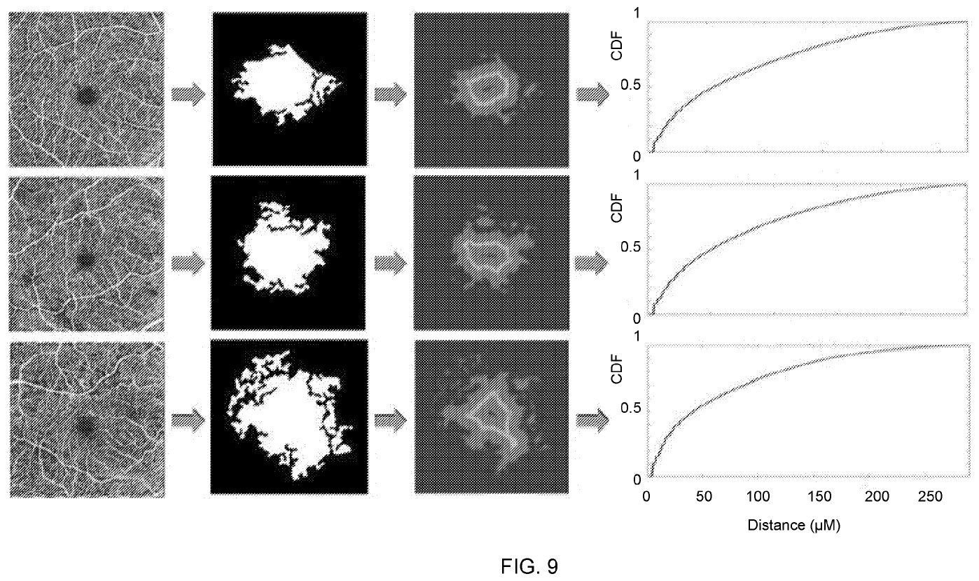

The FAZ Distance Map: FAZ is defined as the dark area in the center of the macula that has no blood vessels. The size of the FAZ can be used as a marker of visual acuity. DR patients typically lose capillaries, resulting in an enlarged FAZ. There is a correlation between the size of FAZ and the severity of DR. FAZ enlargement is one of the earliest changes in the retina caused by DM, so precise measuring and monitoring of the FAZ is useful in early detection of DR. The region growing technique was used to segment the FAZ from the OCTA segmented images. The used dataset is centered around the macula and the center of the image (r_{seed}) is used as a seed point for the technique. A set of morphological filters are used after applying the region growing technique to remove any discontinuity and to fill the holes in the segmented area. To smooth the segmented FAZ, a median filter is utilized. After FAZ segmentation, it is represented in terms of a distance map for input into the classifier. The Euclidean distance is utilized to calculate the distance map between each pixel in the segmented FAZ to its nearest boundary pixel. Then, each one of these calculated distances is represented as a CDF curve, which has 0.03 as an incremental value. FIG. 9 illustrates the OCTA image of the retina, segmented FAZ, distance map of the FAZ, and CDF curves of the distance map for normal (top row), mild NPDR (middle row), and moderate NPDR (bottom row) cases.

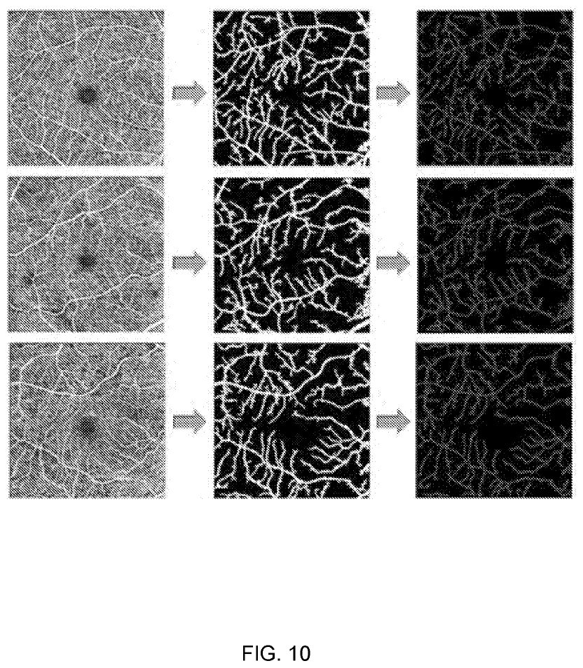

Bifurcation and Crossover Points: Bifurcation, branching, and crossover points of the vessels can be used as landmarks in retinal images, as lower than average numbers of these features are indicative of DR. The bifurcation point are generally T-shaped junctions where a retinal blood vessel splits in two. To segment the vessels, the segmented scan is multiplied by the original scan then stratified by a threshold. A thinning technique is next used to extract the vessels' skeleton and erase the border's pixels. The thinning technique ceases when vessel thickness decreased to a single pixel to maintain connectivity. Then, a filter is applied to delete the points shorter than a given threshold (the expected maximum blood vessel width in the image). For each point in the produced skeleton, the number of neighborhood pixels is calculated to determine if it is a bifurcation point or not. A bifurcation point in a blood vessel is identified point if the number of surrounding pixels=3. A crossover point is identified if the number of the surrounding pixels=4. To use these points as features, the image is split into 8×8, 16×16, 32×32, 1024×1024 windows. Then, the bifurcation and crossover points numbers are determined for each window. Experimental results found that the 128×128 window produced the best results according to the evaluation metrics discussed below, and the window size was utilized in the disclosed system. FIG. 10 depicts the original OCTA image (left column), segmented large blood vessels (middle column), and identified crossover and bifurcation points (right column) for normal (top row), mild NPDR (middle row), and moderate NPDR (bottom row) cases.

In other embodiments, additional features extracted from OCT and/or OCTA image data may be used in addition to or instead of one or more of the above-discussed features. Such additional features include, but are not limited to, capillary dropout and tortuosity of blood vessels.

Clinical and Demographic Data

In the disclosed system, OCT and OCTA imaging data, clinical data and demographic data are collected for each subject. In some embodiments, demographic data used in the system are the sex and age of the subject. Age and sex are relevant to evaluation of retinal layer reflectivity, as described above, and age itself is a risk factor for DR. Use of other demographic data including, without limitation, ethnicity, socioeconomic status, lifestyle, education, and residence is also within the scope of this invention. In some embodiments, the collected clinical data used in the system are visual acuity, HbA1C (glycated hemoglobin test of average blood sugar level), the presence or absence of hypertension, and the presence or absence of dyslipidemia. However, use of other clinical data including, without limitation, blood pressure, lipid (e.g., HDL, LDL, triglyceride) levels, history of heart disease, cerebrovascular disease, neuropathy, and peripheral vascular disease is also within the scope of this invention. All the clinical and demographic data are preprocessed to normalize the values of the features and to impute the missing values. Then, these preprocessed clinical and demographic biomarkers are input to the classifier together with the extracted imaging features.

DR Diagnosis and Grading

A two-stage RF classification system is used to generate a diagnose based on extracted features from OCTA and OCT scans in addition to the demographic and clinical data. In the first stage, the RF classifier is used to distinguish the normal (no DR) from DR subjects. In the second stage, in cases where the subject retina is classified as indicative of DR, the classifier is utilized to grade the DR, such as, for example, distinguishing mild DR subjects from moderate DR subjects. This machine learning classification and grading system was trained and tested on the calculated features from OCTA, OCT, clinical, and demographic data.

Experimental Results

The developed system has been trained and tested on a dataset collected from 111 subjects (36 for normal, 53 for mild NPDR, and 22 for moderate NPDR). The collected data included OCT and OCTA scans in addition to demographic data (e.g., age and gender) and clinical biomarkers (e.g., HbA1c, hypertension, dyslipidemia prevalence, and edema prevalence). Three different retinal specialists diagnosed participating subjects as either having no DR (normal) or having DR with its corresponding grade. The GT was created and labeled by 3 retinal experts. The majority rule was applied to generate the final GT. Both OCT and OCTA scans were retrieved by using an AngioPlex OCT angiography machine, which is manufactured by ZEISS, which generates a complete OCT B-scan and five different OCTA plexuses. The machine utilized Swept-source OCT (SS-OCT) angiography and micro-angiography (OMAG) that are utilized on an SS-OCT DRI OCT Triton. The size of OCTA images used for training and testing is 1024×1024 pixels, spanning a 6×6 mm² with the fovea in the center. The size of OCT images used for training and testing are 1024×1024 pixels. OCT images are captured as raw greyscale scans with 5 plexuses. The field of view is 2 mm posterior-anterior (P-A) and 6 mm nasal-temporal (N-T), and the slice spacing was 0.25 mm.

The developed system was evaluated by utilizing 5 performance metrics: accuracy (ACC), specificity (Spec.), sensitivity (Sens.), dice similarity coefficient (DSC), and the area under the ROC curve (AUC). ACC presents the ratio of the correctly classified cases to the whole tested cases (Eq. 4). Sens. calculates the ratio of the real positive subjects that are correctly recognized (Eq. 5). Spec. calculates the ratio of the real negative subjects that are correctly recognized (Eq. 6). AUC introduces the expectations of a uniformly drawn random positive, which is ranked a uniformly drawn random negative (Eq. 7). DSC computes the relevant correspondence between two areas concerning their false/true negative and positive values (Eq. 8).

$$\begin{matrix}ACC = \frac{TP + TN}{TP + FP + TN + FN} & ( 4 ) \\ Sens . = \frac{TP}{TP + FN} & ( 5 ) \\ Spec . = \frac{TN}{TN + FP} & ( 6 ) \\ AUC = 0.5 ( \frac{TP}{FN + TP} + \frac{TN}{FP + TN} ) & ( 7 ) \\ DSC = \frac{2 TP}{2 TP + FN + FP} & ( 8 )\end{matrix}$$

where TP stands for true positive, TN stands for true negative, FP stands for false positive, and FN stands for false negative.

To avoid overfitting, the 4-fold and leave one subject out (LOSO) cross-validation techniques were utilized. Also, the developed CAD system performance was compared with four various state-of-the-art techniques in both detection and grading stages. These state-of-the-art techniques are support vector machine (SVM) with the linear kernel, SVM with the cubic kernel, classification tree (CT), and KNN.

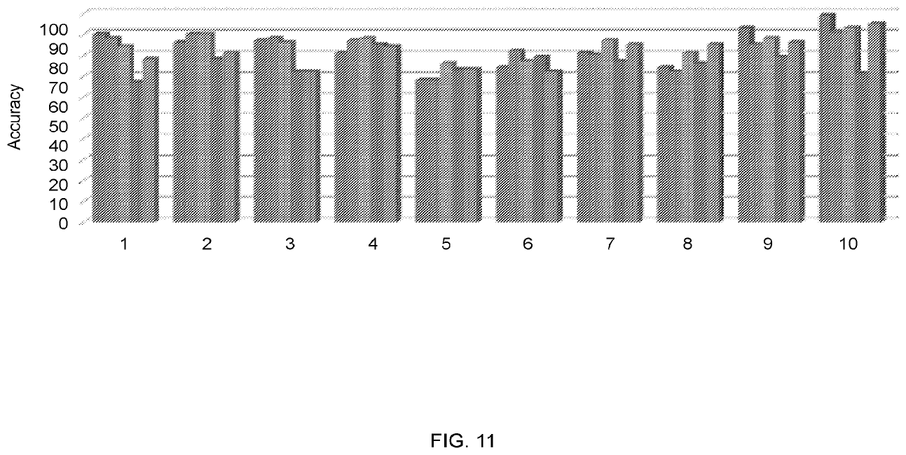

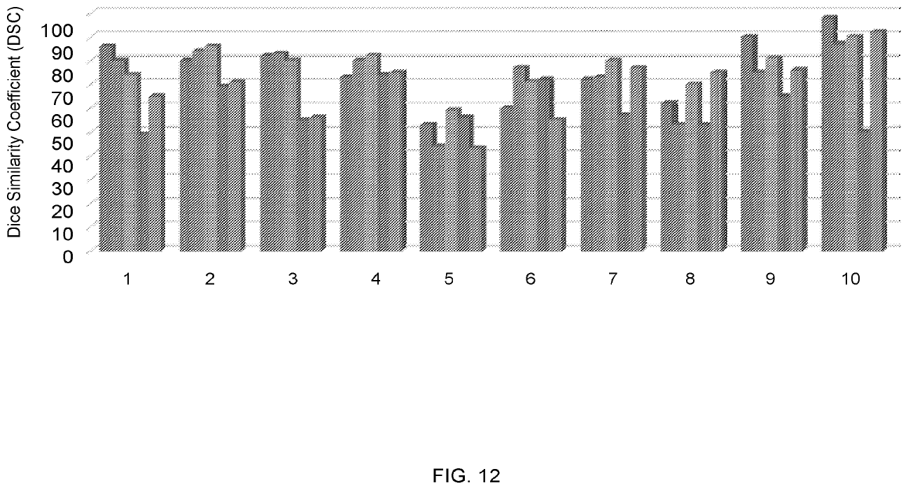

The first stage of classification differentiates normal subjects from DR subjects. Ten various experiments were conducted to evaluate the effect of the extracted features on DR detection in the following combinations: (1) blood vessel density from both SRP and DRP; (2) vessel caliber from both SRP and DRP; (3) FAZ area; (4) number of bifurcation points in the superficial map; (5) curvature of the retinal layers; (6) reflectivity of the retinal layers; (7) thickness of the retinal layers; (8) the three features extracted from OCT scans combined (curvature+reflectivity+thickness); (9) the four features extracted from OCTA scans combined (density+caliber+FAZ+bifurcation); and (10) the three features extracted from OCT scans, the four features extracted from OCTA scans, the clinical data (visual acuity, hypertension, HbA1C, dyslipidemia), and the demographic data (age, gender) in combination. The graphs in FIGS. 11 and 12 respectively show the ACC and DSC of the ten experiments using five different classifiers (in each bar graph, left-to-right, the disclosed RF-based system, SVM (linear), SVM (cubic), KNN, and CT)

As shown in FIGS. 11 and 12, using all the OCT and OCTA features in combination with the clinical and demographic data achieved the highest performance. In the first stage—detection of DR—the disclosed system using a RF classifier achieved a 99% accuracy for both 4-Fold and LOSO cross validation as shown in Table. 1. Since the last scenario achieved the highest accuracy, we used it only in the second stage. In the 2^{nd} stage we wanted to grade the DR cases into mild or moderate. Using a RF classifier with all features, our system achieved an accuracy of 98.7% for both 4-Fold and LOSO as shown in Table 2.

TABLE 1
DR detection performance metrics utilizing different types of classifiers

| Method | Validation | ACC(%) | Sens.(%) | Spec.(%) | DSC(%) | AUC(%) |
|---|---|---|---|---|---|---|
| SVM (Cubic) | 4-Fold | 93 | 93 | 94 | 90 | 93 |
| | LOSO | 95 | 97 | 94 | 92 | 95 |
| SVM | 4-Fold | 91 | 92 | 91 | 87 | 91 |
| (Linear) | LOSO | 90 | 93 | 90 | 85 | 91 |
| KNN | 4-Fold | 71 | 75 | 59 | 50 | 67 |
| | LOSO | 85 | 80 | 88 | 77 | 84 |
| CT | 4-Fold | 95 | 97 | 94 | 92 | 95 |
| | LOSO | 96 | 100 | 94 | 94 | 97 |
| Prop. Sys. | 4-Fold | 99 | 100 | 98 | 98 | 99 |
| (RF) | LOSO | 99 | 100 | 98 | 98 | 99 |

TABLE 2
DR grading performance metrics utilizing different types of classifiers

| Method | Validation | ACC(%) | Sens.(%) | Spec.(%) | DSC(%) | AUC(%) |
|---|---|---|---|---|---|---|
| SVM (Cubic) | 4-Fold | 98.7 | 100 | 98.1 | 97.8 | 99.0 |
| | LOSO | 97.3 | 100 | 96.3 | 95.5 | 98.1 |
| SVM | 4-Fold | 96.0 | 88.0 | 100 | 93.6 | 94.0 |
| (Linear) | LOSO | 96.0 | 88.0 | 100 | 93.6 | 94.0 |
| KNN | 4-Fold | 96.0 | 100 | 94.5 | 93.0 | 97.3 |
| | LOSO | 97.3 | 100 | 96.3 | 95.5 | 98.1 |
| CT | 4-Fold | 94.7 | 91.3 | 96.2 | 91.3 | 93.7 |
| | LOSO | 90.7 | 80.8 | 95.9 | 85.7 | 88.3 |
| Prop. Sys. | 4-Fold | 98.7 | 100 | 97.8 | 99.0 | 98.1 |
| (RF) | LOSO | 98.7 | 100 | 97.8 | 99.0 | 98.2 |

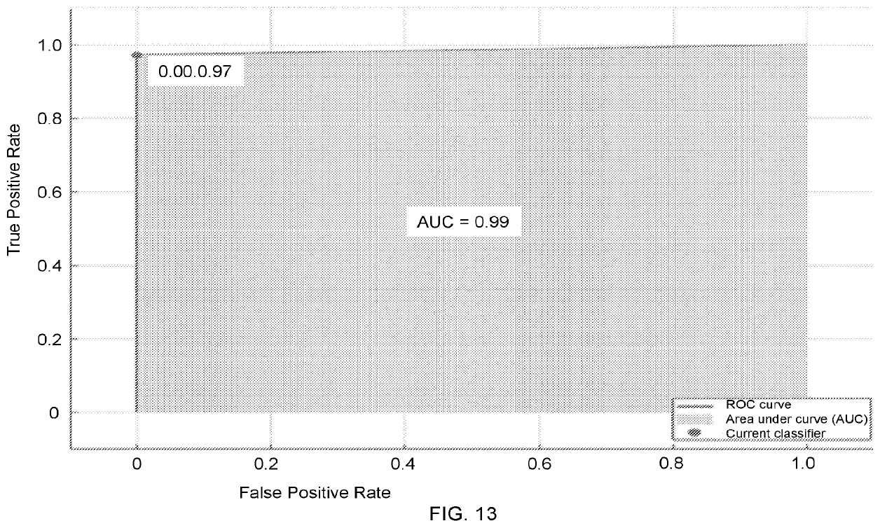

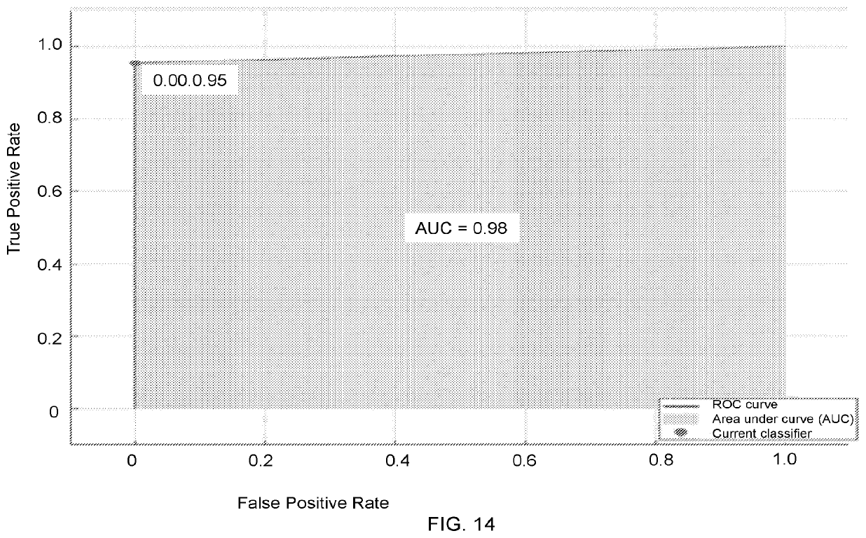

The accuracy of the disclosed system is further evaluated against the classification threshold selection by utilizing the receiver operating characteristic (ROC) curve. This experiment is important to test the robustness of the used classifier, that is, the ability of the classifier to make correct predictions from noisy data. FIG. 13 illustrates the ROC curve for the classifier with the highest performance in the detection stage, which is the RF classifier. FIG. 14 illustrates the ROC curve for the classifier with the highest performance in the grading stage, which is also the RF classifier.

Further statistical analysis of the disclosed system included testing additional combinations of input data by four-fold cross validation and leave-one-subject-out (LOSO) validation. These results were then compared to the clinical grading of DR, which was considered the gold standard. The accuracy, sensitivity, specificity, dice similarity coefficient, and area under the curve of the system were calculated with use of OCT data alone, OCTA data alone, combined OCT and OCTA data, and finally combined OCT, OCTA, clinical, and demographic data.

The first stage of the classifier system classifies images as demonstrating DR or no DR. In this first stage, the system was tested with three different sets of data inputs: OCT data alone, OCTA data alone, or OCT, OCTA, demographic, and clinical data combined. Combining all data produced the best results, with diagnostic accuracy of 97-98% and an AUC of 0.981 by LOSO and 0.987 by four-fold cross validation (Table 3). AUCs for OCT data alone were approximately 0.89, for combined OCT and OCTA 0.968, and for OCT, OCT, and clinical and demographic data 0.987.

TABLE 3
Performance of the system for stage
1, distinguishing DR from no DR

| Features | ACC(%) | Sens. (%) | Spec. (%) | DSC(%) | AUC |
|---|---|---|---|---|---|
| OCT | 86.5 | 92.3 | 84.8 | 76.2 | 0.885 |
| OCTA | 94.6 | 87.8 | 98.6 | 92.3 | 0.932 |
| OCT + OCTA | 95.5 | 100 | 93.7 | 92.5 | 0.968 |
| All features | 98.2 | 100 | 97.4 | 97.2 | 0.987 |

The second stage of the classifier system grades the level of NPDR in those images identified as having DR in stage 1. The system was again tested four times, first with data from OCT images alone (OCT), second from OCTA images alone (OCTA), third from images of both modalities (OCT+OCTA), and finally with all imaging, clinical, and demographic data (all features). Using all features as input performed the best in all metrics. No cases of severe NPDR were included in the dataset, so the two outputs were either mild or moderate NPDR. By both LOSO and four-fold cross validation, the system's accuracy for the grading stage was 98.7%, sensitivity 100%, specificity 97.8%, DSC 99%, and AUC 0.981 (Table 4). Again, a steady improvement in all metrics was observed when the system was given only OCT data, to OCT and OCTA data, to OCT, OCTA, clinical, and demographic data, with AUCs increasing from 0.897, to 0.967, to 0.981 (Table 4).

TABLE 4
Performance of the system for stage 2, grading
images identified as having DR in stage 1

| Classifiers | ACC(%) | Sens. (%) | Spec. (%) | DSC(%) | AUC |
|---|---|---|---|---|---|
| OCT | 88.3 | 92.5 | 86.9 | 79.3 | 0.897 |
| OCTA | 94.7 | 91.3 | 96.2 | 91.3 | 0.937 |
| OCT + OCTA | 97.3 | 95.4 | 98.1 | 95.4 | 0.967 |
| All Features | 98.7 | 100 | 97.8 | 99.0 | 0.981 |

The overall performance of the automated diagnostic system involves the combined results of stage 1 (DR vs no DR) and stage 2 (grading of NPDR) in sequence. As before, the system was tested with four different data sets: OCT images alone (OCT), OCTA images alone, both OCT and OCTA images (OCT+OCTA), and all imaging, clinical, and demographic data (all features). All features again performed the best in all metrics. When running the whole system on all data inputs, final accuracy was 96%, sensitivity 100%, specificity 94%, DSC 98%, and AUC 0.960 (Table 5). When the system was given OCT data only, AUC was 0.783, increasing to 0.921 with combined OCT and OCTA data, and finally 0.960 when given all data modalities (OCT, OCTA, clinical, and demographic data).

TABLE 5
Overall performance of the system

| Classifiers | ACC(%) | Sens. (%) | Spec. (%) | DSC(%) | AUC |
|---|---|---|---|---|---|
| OCT | 75.6 | 84.6 | 86.9 | 72.2 | 0.783 |
| OCTA | 88.3 | 79.2 | 94.1 | 83.7 | 0.865 |
| OCT + OCTA | 92.2 | 95.4 | 98.1 | 91.1 | 0.921 |
| All Features | 96.0 | 100 | 94.1 | 98.0 | 0.960 |

The disclosed CAD system for the diagnosis and grading of NPDR integrates imaging data from both OCT and OCTA with basic clinical and demographic data. When utilized with 111 patients, the AUC of the final diagnosis was 0.76 when analyzing structural OCT data alone, improved to 0.92 with the addition of OCT angiographic data, and improved further to 0.96 with the addition of clinical and demographic data. In certain embodiments, the CAD system is embodied in a non-transitory computer readable storage medium having computer program instructions stored thereon that, when executed by a processor, cause the processor to perform the instructions to classify a subject retina as normal or DR, and if DR, to grade the DR, based on the input features extracted from image data, demographic data, and clinical data.

Two points stand out from the results of the disclosed system. First, OCT angiography imaging clearly adds significant value to an automated diagnostic system for DR. DR is primarily a disease of the retinal vasculature, and OCTA provides instructive information about the status of the vasculature that structural OCT does not. In the disclosed system, the size of the FAZ and density of capillaries within the macula are both known to have diagnostic value in diagnosing DR, consistent with the pathophysiology of the disease, driving capillary nonperfusion and eventually macular ischemia. OCTA's ability to image both the deep and superficial vascular plexuses—both affected in DR—its noninvasive methods, ease of acquisition, and presence on the same imaging platforms as OCT are distinct advantages over fluorescein angiogram, and make it a complement to conventional OCT.

Second, the addition of simple clinical and demographic data also had added value for the system, improving the first stage, second stage, and overall performance by 2-4% of AUC. Systemic hypertension and hemoglobin A1c are perhaps the oldest and most reliable predictors of DR onset and progression. While these risk factors are well known, what was unclear was to what extent, if any, this additional information might add diagnostic value. If the local effects of poor blood glucose and blood pressure control are already indirectly captured by OCTA imaging of the retinal vasculature, for instance, one would not expect these data to add any significant value. However, providing inputs of the patient's age, gender, last hemoglobin A1c, and history of systemic hypertension and/or hyperlipidemia provided a small but appreciable improvement in diagnostic performance, increasing AUC from 0.92 to 0.96.

The disclosed software can analyze both superficial and deep retinal maps from OCTA scans. Also, the software can analyze the OCT scans to retrieve features of retinal layers. The extracted features from OCTA and OCT scans are integrated with the clinical and demographic biomarkers for the patient to create a comprehensive diagnostic system. On the other hand, the software can measure four different retinal vasculature features, which are blood vessel density, blood vessel caliber, foveal avascular zone area, and bifurcation and crossover points. It also can extract three main retinal layers features, which are thickness, reflectivity, and curvature. In other embodiments, additional retinal layer and retinal vascular features may be used in addition to or instead of the above listed features, these additional features including, but not limited to, capillary dropout and tortuosity of vessels.

Various aspects of different embodiments of the present disclosure are expressed in paragraphs X1, X2, and X3 as follows:

X1: One embodiment of the present disclosure includes a computer-implemented method for diagnosing diabetic retinopathy, the method comprising receiving image data including a retina of a subject; processing the image data to segment the retina; extracting at least one feature from the segmented retina; receiving demographic data and clinical data associated with the subject; and generating, using a machine learning classifier, a diagnosis for the subject based at least in part on the at least one feature, the demographic data, and the clinical data.

X2: Another embodiment of the present disclosure includes a computer-implemented method for classifying a retina, the method comprising processing image data including a subject retina to segment the subject retina; extracting at least one feature from the segmented retina; receiving demographic data and clinical data associated with the subject retina; and classifying, using a machine learning classifier, the subject retina as normal or indicative of diabetic retinopathy based at least in part on the at least one feature, the demographic data, and the clinical data.

X3: A further embodiment of the present disclosure includes a non-transitory computer readable storage medium having computer program instructions stored thereon that, when executed by a processor, cause the processor to perform the following instructions: receiving at least one feature extracted from OCA image data of a subject retina; receiving at least one feature extracted from OCTA image data of the subject retina; receiving demographic data and clinical data associated with the subject retina; classifying the subject retina as normal or indicative of diabetic retinopathy based at least in part on the at least one feature, the demographic data, and the clinical data.

Yet other embodiments include the features described in any of the previous paragraphs X1, X2, or X3 combined with one or more of the following aspects:

Wherein the diagnosis is one of normal and diabetic retinopathy.

Wherein the diagnosis is one of normal, mild nonproliferative diabetic retinopathy, moderate nonproliferative diabetic retinopathy, severe nonproliferative diabetic retinopathy, and proliferative diabetic retinopathy.

Wherein the diagnosis is one of normal, mild nonproliferative diabetic retinopathy, and moderate nonproliferative diabetic retinopathy.

Wherein the image data includes optical coherence tomography (OCT) image data and optical coherence tomography angiography (OCTA) image data.

Wherein processing the image data to segment the subject retina includes processing the OCT image data to segment the subject retina into a plurality of retinal layers.

Wherein the at least one feature is at least one of retinal layer thickness, reflectivity, and curvature.

Wherein processing the image data to segment the subject retina includes processing the OCTA image data to segment vasculature of the subject retina.

Wherein the at least one feature is at least one of bifurcation points, crossover points, distance map of the foveal avascular zone, blood vessel density, and blood vessel caliber.

Wherein the demographic data includes at least one of sex and age.

Wherein the clinical data includes at least one of visual acuity, hypertension, HbA1C, and dyslipidemia.

Wherein the classifier is a random forest classifier.

Wherein the classifier is a two-stage classifier.

Wherein the two-stage classifier includes a first stage which generates a diagnosis of normal or diabetic retinopathy; and a second stage which, if the first stage diagnoses diabetic retinopathy, generates a diagnosis grading the diabetic retinopathy.

Wherein the image data includes optical coherence tomography (OCT) image data and optical coherence tomography angiography (OCTA) image data.

Wherein processing the image data to segment the subject retina includes processing the OCT image data to segment the subject retina into a plurality of retinal layers.

Wherein processing the image data to segment the subject retina includes processing the OCTA image data to segment vasculature of the subject retina.

Wherein the at least one feature extracted from OCA image data of the subject retina is extracted from OCA image data of the subject retina segmented into a plurality of retinal layers and wherein the at least one feature extracted from OCTA image data of the subject retina is extracted from OCTA image data of a segmented vasculature of the subject retina.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1) A computer-implemented method for diagnosing diabetic retinopathy, the method comprising:
receiving image data including a retina of a subject;
processing the image data to segment the retina;
extracting at least one feature from the segmented retina;
receiving demographic data and clinical data associated with the subject; and
generating, using a machine learning classifier, a diagnosis for the subject based at least in part on the at least one feature, the demographic data, and the clinical data.

2) The method of claim 1, the diagnosis is one of normal and diabetic retinopathy.

3) The method of claim 1, wherein the diagnosis is one of normal, mild nonproliferative diabetic retinopathy, moderate nonproliferative diabetic retinopathy, severe nonproliferative diabetic retinopathy, and proliferative diabetic retinopathy.

4) The method of claim 3, wherein the diagnosis is one of normal, mild nonproliferative diabetic retinopathy, and moderate nonproliferative diabetic retinopathy.

5) The method of claim 1, wherein the image data includes optical coherence tomography (OCT) image data and optical coherence tomography angiography (OCTA) image data.

6) The method of claim 5, wherein processing the image data to segment the subject retina includes processing the OCT image data to segment the subject retina into a plurality of retinal layers.

7) The method of claim 6, wherein the at least one feature is at least one of retinal layer thickness, reflectivity, and curvature.

8) The method of claim 5, wherein processing the image data to segment the subject retina includes processing the OCTA image data to segment vasculature of the subject retina.

9) The method of claim 8, wherein the at least one feature is at least one of bifurcation points, crossover points, distance map of the foveal avascular zone, blood vessel density, and blood vessel caliber.

10) The method of claim 1, wherein the demographic data includes at least one of sex and age.

11) The method of claim 1, wherein the clinical data includes at least one of visual acuity, hypertension, HbA1C, and dyslipidemia.

12) The method of claim 1, wherein the classifier is a random forest classifier.

13) The method of claim 1, wherein the classifier is a two-stage classifier.

14) The method of claim 13, wherein the two-stage classifier includes
a first stage which generates a diagnosis of normal or diabetic retinopathy; and
a second stage which, if the first stage diagnoses diabetic retinopathy, generates a diagnosis grading the diabetic retinopathy.

15) A computer-implemented method for classifying a retina, the method comprising:
processing image data including a subject retina to segment the subject retina;
extracting at least one feature from the segmented retina;
receiving demographic data and clinical data associated with the subject retina; and
classifying, using a machine learning classifier, the subject retina as normal or indicative of diabetic retinopathy based at least in part on the at least one feature, the demographic data, and the clinical data.

16) The method of claim 15, wherein the image data includes optical coherence tomography (OCT) image data and optical coherence tomography angiography (OCTA) image data.

17) The method of claim 16, wherein processing the image data to segment the subject retina includes processing the OCT image data to segment the subject retina into a plurality of retinal layers.

18) The method of claim 16, wherein processing the image data to segment the subject retina includes processing the OCTA image data to segment vasculature of the subject retina.

19) A non-transitory computer readable storage medium having computer program instructions stored thereon that, when executed by a processor, cause the processor to perform the following instructions:
receiving at least one feature extracted from OCA image data of a subject retina;
receiving at least one feature extracted from OCTA image data of the subject retina;
receiving demographic data and clinical data associated with the subject retina;
classifying the subject retina as normal or indicative of diabetic retinopathy based at least in part on the at least one feature, the demographic data, and the clinical data.

20) The non-transitory computer readable storage medium of claim 19,
wherein the at least one feature extracted from OCA image data of the subject retina is extracted from OCA image data of the subject retina segmented into a plurality of retinal layers and
wherein the at least one feature extracted from OCTA image data of the subject retina is extracted from OCTA image data of a segmented vasculature of the subject retina.
